(19) United States
(12) Patent Application Publication
(10) Pub. No.: US 2026/0307079 A1
(43) Pub. Date: Oct. 8, 2026

(54) INTEGRATED DEVICE FOR NUCLEIC ACID EXTRACTION, AMPLIFICATION AND DETECTION

(71) Applicant: HEFEI DAHUI GENE TECHNOLOGY CO., LTD, Hefei (CN)

(72) Inventors: Zhizhen Xu, Hefei (CN); Dawei Fang, Hefei (CN); Yi Xia, Hefei (CN); Nan Cheng, Hefei (CN)

(21) Appl. No.: 19/681,083

(22) Filed: May 18, 2026

Related U.S. Application Data
(63) Continuation of application No. PCT/CN2024/076853, filed on Feb. 8, 2024.

(30) Foreign Application Priority Data
Dec. 20, 2023 (CN) 202311765329.2

(51) Int. Cl. B01L 7/00; B01L 3/00; B01L 7/04; B01L 9/06; G01N 21/64

(52) U.S. Cl. CPC B01L 7/52; B01L 3/5082; B01L 7/04; B01L 9/06; G01N 21/6428; G01N 21/645; B01L 2200/0631; B01L 2200/0647; B01L 2200/10; B01L 2300/0654; B01L 2300/0832; B01L 2300/1805; B01L 2300/1883; B01L 2400/043; G01N 2021/6471

(57) ABSTRACT

An integrated device for nucleic acid extraction, amplification and detection, including a support, an amplification unit, an extraction unit, a fluorescence detection unit, and a driving unit. The amplification unit is disposed on the support. The extraction unit is mounted on the amplification unit. The extraction unit includes a test tube mounting assembly, a first driving member, and a magnetic member, where the test tube mounting assembly is provided with a first mounting hole, the magnetic member is arranged in a wall of the first mounting hole, and the first driving member is used to drive the test tube mounting assembly to rotate, thereby driving the magnetic member to rotate around an outer periphery of a test tube located in the test tube mounting assembly for nucleic acid extraction. The fluorescence detection unit is mounted on the support, and used to perform fluorescence detection on the test tube.

### CROSS REFERENCE TO RELATED APPLICATIONS

The present application is a Continuation Application of PCT Application No. PCT/CN2024/076853 filed on Feb. 8, 2024, which claims the benefit of Chinese Patent Application No. 202311765329.2 filed on Dec. 20, 2023. All the above are hereby incorporated by reference in their entirety.

### TECHNICAL FIELD

The present disclosure relates to the field of nucleic acid technology, and in particular to an integrated device for nucleic acid extraction, amplification and detection.

### BACKGROUND

Currently, processes of nucleic acid extraction, amplification and detection are all performed manually in a laboratory. Performing a nucleic acid test requires a plurality of steps including extraction, polymerase chain reaction (PCR) amplification, and fluorescence detection. In order to prevent aerosol contamination and ensure biosafety, the existing processes of nucleic acid extraction, PCR amplification and detection are performed in separate laboratory zones, which requires the use of a plurality of devices, such as a nucleic acid extractor and a PCR amplification instrument, each being a standalone product. One person must perform different operational steps and processes across multiple independent zones and different devices. This increases the complexity and tediousness of the nucleic acid extraction, PCR amplification and detection processes, making human errors during manual operation inevitable. At the same time, the number of nucleic acid tests a person can perform at one time is small, and the time required is long. This further leads to low manual operation efficiency. Performing different steps in different laboratory zones also fails to adequately prevent aerosol contamination, resulting in low safety.

### SUMMARY

The technical problem to be solved by the present disclosure is that nucleic acid extraction, amplification and detection are performed in separate laboratory zones, resulting in a cumbersome process, low operational efficiency, and inadequate prevention of aerosol contamination.

In order to solve the above technical problem, the present disclosure provides an integrated device for nucleic acid extraction, amplification and detection, including:

a support;

an amplification unit, disposed on the support, and configured to perform temperature regulation;

an extraction unit, mounted on the amplification unit, where the extraction unit includes a test tube mounting assembly, a first driving member and a magnetic member, the test tube mounting assembly is provided with a first mounting hole, the magnetic member is arranged in a wall of the first mounting hole, and the first driving member is mounted on the support, and configured to drive the test tube mounting assembly to rotate, thereby driving the magnetic member to rotate around an outer periphery of a test tube located in the test tube mounting assembly and perform nucleic acid extraction;

a fluorescence detection unit, mounted on the support, and configured to perform fluorescence detection on the test tube; and

a driving unit, mounted on the support, where an output end of the driving unit connects to the amplification unit, and is configured to drive the amplification unit and the extraction unit to rotate relative to the fluorescence detection unit.

In some embodiments, the extraction unit further includes a gear plate, and an output shaft of the first driving member passes through the amplification unit and connects to the gear plate; and

the test tube mounting assembly includes a gear member, where the gear member is provided with the first mounting hole, the gear plate is meshed with the gear member, and the magnetic member is arranged on the gear member.

In some embodiments, the first mounting hole passes through two ends of the gear member, a second mounting hole is provided in the wall of the first mounting hole, and the magnetic member is arranged in the second mounting hole.

In some embodiments, the extraction unit further includes a bottom plate and a top plate, an accommodating cavity is formed between the bottom plate and the top plate, the gear plate is arranged in the accommodating cavity, the bottom plate is arranged on the amplification unit, and the bottom plate is provided with a third mounting hole through which a bottom of the test tube passes.

In some embodiments, an end surface of the gear member facing the bottom plate is provided with a fourth mounting hole, an end surface of the gear member facing the top plate is provided with a fifth mounting hole, and each of the fourth mounting hole and the fifth mounting hole comprises a ball;

the test tube mounting assembly further includes a matching member, where the matching member is arranged on the bottom plate, a first slide groove is provided on a side of the matching member facing the top plate, and the third mounting hole passes through the first slide groove; a second slide groove corresponding to the gear member is provided on a side of the top plate facing the bottom plate;

the gear member is arranged between the top plate and the matching member, the gear member and the matching member are arranged correspondingly, and the gear member is configured to rotate in the first slide groove and the second slide groove via the balls.

In some embodiments, the test tube mounting assembly further includes a first heating member, and the first heating member is arranged on the top plate, corresponding to the gear member.

In some embodiments, the test tube mounting assembly further includes a heat insulating member, and the heat insulating member is sleeved on an outer periphery of the first heating member.

In some embodiments, a plurality of the test tube mounting assemblies are provided, and the plurality of the test tube mounting assemblies are arranged around a peripheral side of the gear plate to form a test tube mounting assembly group;

where n test tube mounting assembly groups are provided and the n test tube mounting assembly groups are arranged in sequence along a radial direction of the gear plate, where n ≥ 1.

In some embodiments, the amplification unit includes a heating plate, a second heating member and a heat conductive member, the second heating member is arranged on the heating plate and located below the extraction unit, and the heat conductive member and the first driving member are arranged on a side of the heating plate away from the second heating member.

In some embodiments, a plurality of the second heating members are provided, and the plurality of the second heating members are spliced around an axis of the heating plate to form a ring.

In some embodiments, the integrated device for nucleic acid extraction, amplification and detection further includes a cooling fan, where the cooling fan is mounted on the support, and a suction port of the cooling fan faces the amplification unit.

In some embodiments, a light guiding unit is arranged between the extraction unit and the amplification unit, the light guiding unit is provided with a sixth mounting hole and a first light guiding hole, the sixth mounting hole and the third mounting hole are arranged correspondingly, the bottom of the test tube extends into the light guiding unit through the sixth mounting hole, and light emitted from the fluorescence detection unit passes through the first light guiding hole and is directed onto the bottom of the test tube.

In some embodiments, the light guiding unit includes a light guiding plate and a light guiding pillar, the first light guiding hole is arranged on a side of the light guiding plate facing the fluorescence detection unit to make the light emitted from the fluorescence detection unit to pass through, the sixth mounting hole is arranged on a side of the light guiding plate facing the extraction unit, the light guiding pillar is arranged in the light guiding plate and is located between the first light guiding hole and the sixth mounting hole, and the light guiding pillar is configured to direct the light emitted from the fluorescence detection unit onto the bottom of the test tube through the first light guiding hole.

In some embodiments, a center line of the first light guiding hole is arranged perpendicular to a center line of the sixth mounting hole.

In some embodiments, a plurality of the first light guiding holes are provided, and the plurality of first light guiding holes are arranged at intervals around an axis of the light guiding plate;

a plurality of the sixth mounting holes are provided, the plurality of the sixth mounting holes are arranged at intervals around the axis of the light guiding plate, and the plurality of the first light guiding holes and the plurality of the sixth mounting holes are arranged in a one-to-one correspondence;

the light guiding pillar is disposed between each of the plurality of the first light guiding holes and a corresponding one of the plurality of the sixth mounting holes.

In some embodiments, the light guiding plate further includes a light guiding tube, one end of the light guiding tube is in communication with the first light guiding hole, another end of the light guiding tube is in communication with the sixth mounting hole, and the light guiding pillar is arranged in the light guiding tube.

In some embodiments, the fluorescence detection unit includes an emission end;

a diameter of the light guiding plate is smaller than a diameter of the extraction unit, and the diameter of the light guiding plate is smaller than a diameter of the amplification unit, a groove is defined among the extraction unit, the light guiding plate, and the amplification unit, and the emission end is located in the groove.

In some embodiments, the fluorescence detection unit includes a light emitting assembly, an optical fiber, and an optical fiber emitter, the light emitting assembly and the optical fiber emitter are mounted on the support, the light emitting assembly connects to the optical fiber emitter through the optical fiber, the optical fiber emitter is provided with the emission end, and the light emitted from the emission end is directed onto the bottom of the test tube through the first light guiding hole and the light guiding pillar.

In some embodiments, a plurality of the light emitting assemblies are provided, and the plurality of the light emitting assemblies are arranged at intervals around an output shaft of the driving unit;

a plurality of the optical fiber emitters are provided, and the plurality of the optical fiber emitters are arranged at intervals around an axis of the support.

In some embodiments, the fluorescence detection unit further includes a driving assembly, the light emitting assembly includes a plurality of light sources, a plurality of filters and a first transmission member, the first transmission member is provided with a transmission end connected to the driving assembly and a mounting end located on an emission optical path, the plurality of light sources are mounted on the mounting end at intervals around an axis of the mounting end, and each of the plurality of filters is correspondingly provided in front of an optical emission head of a corresponding one of the plurality of light sources;

in response to that the driving assembly drives the first transmission member to rotate once, the first transmission member drives one of the plurality of light sources to rotate to the emission optical path to emit light with a corresponding wavelength.

In some embodiments, the driving assembly includes a second driving member and a conveyor belt, and the first transmission member and the second driving member are synchronously driven through the conveyor belt.

In some embodiments, the driving unit includes a third driving member, a synchronous belt, and a second transmission member, the third driving member is mounted on the support, the second transmission member is mounted on a bottom of the amplification unit, and the third driving member and the second transmission member are synchronously driven through the synchronous belt to drive the amplification unit to rotate.

Compared with the prior art, the integrated device for nucleic acid extraction, amplification and detection according to an embodiment of the present disclosure has the following beneficial effects:

The extraction unit of the embodiment of the present disclosure is used to extract nucleic acids from a test tube placed in a placement hole, during the extraction, the first driving member is used to drive the magnetic member to rotate around an outer periphery of the test tube placed in the first mounting hole, and the magnetic member arranged outside the first mounting hole provides a magnetic attraction force to attract magnetic beads in the test tube, driving the magnetic beads to rotate, so as to perform the nucleic acid extraction. The amplification unit is used to regulate the temperature of the bottom of the test tube to perform nucleic acid amplification. The driving unit drives the test tube in the extraction unit to rotate relative to the fluorescence detection unit, so that the fluorescence detection unit can perform the fluorescence detection on the test tube.

In the present disclosure, by integrating the functions of nucleic acid extraction, amplification and fluorescence detection into a single device, thereby eliminating the need to perform nucleic acid extraction, amplification and fluorescence detection operations in separate laboratory zones. This not only improves efficiency but further shortens detection time, and avoids the problem of aerosol contamination caused by transporting samples to different zones for operation.

### BRIEF DESCRIPTION OF THE DRAWINGS

FIG. 1 is a schematic structural diagram of an integrated device for nucleic acid extraction, amplification and detection according to an embodiment of the present disclosure.

FIG. 2 is a partially exploded view of an integrated device for nucleic acid extraction, amplification and detection according to an embodiment of the present disclosure from a first angle.

FIG. 3 is an exploded schematic diagram of a magnetic member and a gear member according to an embodiment of the present disclosure.

FIG. 4 is an exploded view of an integrated device for nucleic acid extraction, amplification and detection according to an embodiment of the present disclosure.

FIG. 5 is an exploded view of an amplification unit, an extraction unit, and a light guiding unit according to an embodiment of the present disclosure from a first angle.

FIG. 6 is an exploded view of an amplification unit, an extraction unit, and a light guiding unit according to an embodiment of the present disclosure from a second angle.

FIG. 7 is an exploded schematic diagram of an extraction unit according to an embodiment of the present disclosure.

FIG. 8 is an exploded schematic diagram of a top plate, a gear plate and a gear member according to an embodiment of the present disclosure.

FIG. 9 is an exploded view of a top plate, a first heating member, and a heat insulating member according to an embodiment of the present disclosure.

FIG. 10 is a partially enlarged view of the portion A circled in FIG. 7 according to an embodiment of the present disclosure.

FIG. 11 is a partially enlarged view of the portion B circled in FIG. 7 according to an embodiment of the present disclosure.

FIG. 12 is a partially enlarged view of the portion C circled in FIG. 9 according to an embodiment of the present disclosure.

FIG. 13 is an exploded view of a driving unit and an amplification unit according to an embodiment of the present disclosure.

FIG. 14 is a schematic structural diagram of an amplification unit, an extraction unit, and a light guiding unit according to an embodiment of the present disclosure.

FIG. 15 is a partially exploded view of a support, a fluorescence detection unit, and a cooling fan according to an embodiment of the present disclosure.

FIG. 16 is a partially exploded view of an integrated device for nucleic acid extraction, amplification and detection according to an embodiment of the present disclosure from a second angle.

FIG. 17 is an exploded schematic diagram of a light guiding plate according to an embodiment of the present disclosure.

FIG. 18 is a partially enlarged view of the portion D circled in FIG. 2 according to an embodiment of the present disclosure.

In the drawings, 1, support; 11, top support; 12, bottom support;

2, amplification unit; 21, heating plate; 2101, third through-hole; 22, second heating member; 23, heat conductive member;

3, extraction unit; 31, test tube mounting assembly; 3101, first mounting hole; 3102, gear member; 31021, second mounting hole; 31022, fourth mounting hole; 31023, fifth mounting hole; 31024, main body; 31025, gear tooth; 3103, matching member; 31031, first slide groove; 3104, first heating member; 3105, heat insulating member; 31051, first base; 31052, first mounting groove; 31053, first connecting member; 310531, second base; 310532, second mounting groove; 32, first driving member; 33, magnetic member; 34, gear plate; 3401, first through-hole; 35, bottom plate; 3501, third mounting hole; 3502, second through-hole; 36, top plate; 3601, second slide groove; 37, second connecting member;

4, fluorescence detection unit; 41, light emitting assembly; 4101, first transmission member; 42, optical fiber; 43, optical fiber emitter; 44, driving assembly; 4401, second driving member; 4402, conveyor belt;

5, driving unit; 51, third driving member; 52, synchronous belt; 53, second transmission member;

6, cooling fan;

7, light guiding unit; 71, sixth mounting hole; 72, first light guiding hole; 73, light guiding plate; 7301, light guiding tube; 73011, light guiding part; 7302, first plate; 7303, second plate; 74, light guiding pillar; 75, groove.

### DETAILED DESCRIPTION

The specific embodiment of the present disclosure is further described in detail below in conjunction with the drawings and examples. The following examples are provided to illustrate the present disclosure, but are not intended to limit the scope of the present disclosure.

As shown in FIG. 1, an embodiment of the present disclosure provides an integrated device for nucleic acid extraction, amplification and detection, which is used for nucleic acid extraction, amplification and fluorescence detection. In actual application, a test tube is placed in the integrated device for nucleic acid extraction, amplification and detection, and the integrated device is used to perform nucleic acid extraction, amplification and fluorescence detection on a nucleic acid solution in the test tube.

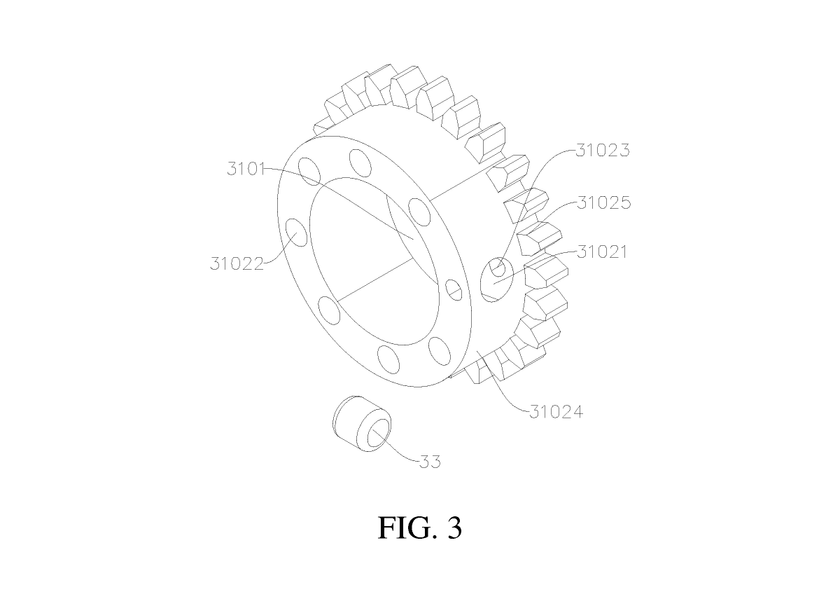

As can be seen from FIGS. 1-3 and 5, the integrated device for nucleic acid extraction, amplification and detection includes a support 1, an amplification unit 2, an extraction unit 3, a fluorescence detection unit 4, and a driving unit 5. The support 1 in this embodiment serves as a mounting structure to carry the amplification unit 2, the extraction unit 3, the fluorescence detection unit 4, and the driving unit 5. The extraction unit 3 is configured to extract nucleic acids from the nucleic acid solution in the test tube. In this embodiment, magnetic beads are placed in the test tube, and fully bind with lysed nucleic acids. The extraction unit 3 includes a test tube mounting assembly 31, a first driving member 32, and a magnetic member 33. The test tube mounting assembly 31 includes a first mounting hole 3101 (as shown in FIG. 3) for placing the test tube. The magnetic member 33 is arranged in a wall of the first mounting hole 3101. The magnetic member 33 provides a magnetic attraction force to attract the magnetic beads in the test tube. The first driving member 32 is mounted on the support 1, and is configured to drive the test tube mounting assembly 31 to rotate, so as to drive the magnetic member 33 to rotate around an outer periphery of the test tube placed in the test tube mounting assembly 31, and the magnetic beads in the test tube rotate accordingly, so that nucleic acids are cleaned and purified in the test tube, completing nucleic acid extraction. The extraction unit 3 is mounted on the amplification unit 2, the amplification unit 2 is configured to regulate a temperature of a bottom of the test tube, to perform the amplification of the nucleic acids in the test tube. In this embodiment, the fluorescence detection unit 4 and the driving unit 5 are mounted on the support 1, and an output end of the driving unit 5 connects to the amplification unit 2. The amplification unit 2 is arranged on the support 1 via the driving unit 5. The driving unit 5 is configured to drive the amplification unit 2 and the extraction unit 3 to rotate relative to the fluorescence detection unit 4, and the fluorescence detection unit 4 emits light onto the test tube to perform fluorescence detection on the nucleic acids in the test tube.

In this embodiment, by integrating the functions of nucleic acid extraction, amplification and fluorescence detection into a single device, thereby eliminating the need to perform nucleic acid extraction, amplification and fluorescence detection operations in separate laboratory zones. This not only improves efficiency but further shortens detection time, and avoids the problem of aerosol contamination caused by transporting samples to different zones for operation.

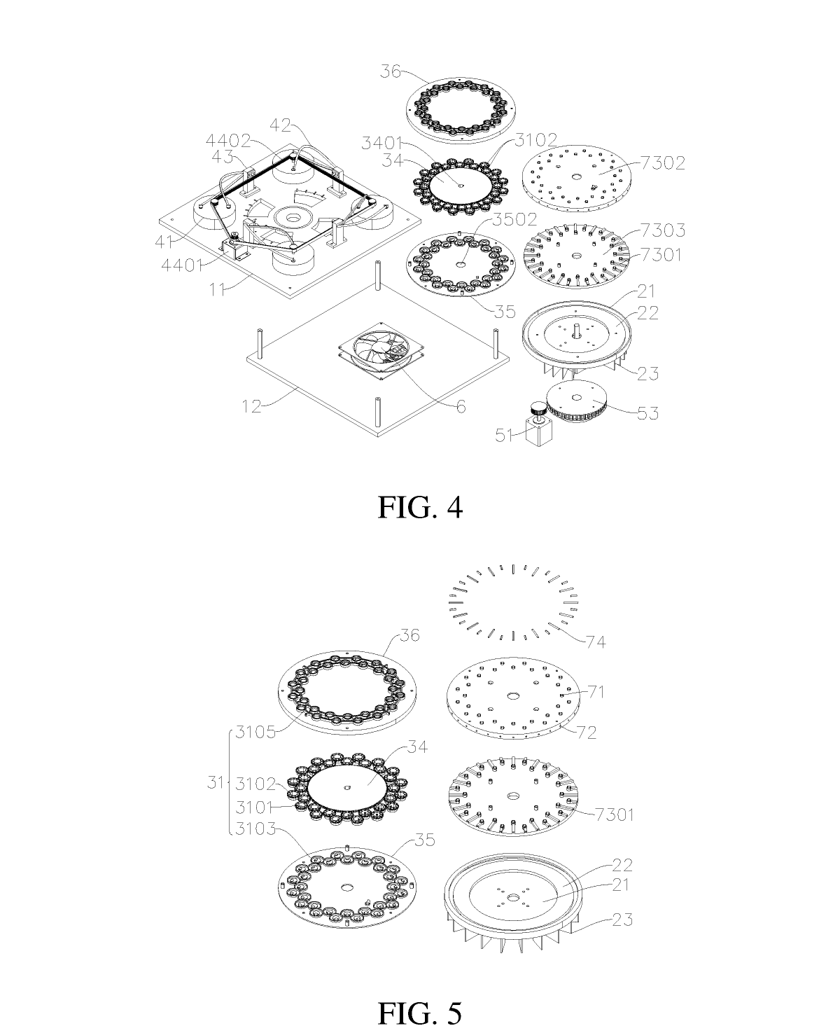

As can be seen from FIG. 4, the extraction unit 3 further includes a gear plate 34, and the gear plate 34 is provided with a first through-hole 3401. An output shaft of the first driving member 32 passes through the amplification unit 2 and is fitted into the first through-hole 3401 of the gear plate 34, so as to realize the connection between the output shaft of the first driving member 32 and the gear plate 34. The test tube mounting assembly 31 includes a gear member 3102, and the gear plate 34 is meshed with the gear member 3102. The gear member 3102 is provided with a first mounting hole 3101, and the magnetic member 33 is arranged in a wall of the first mounting hole 3101 of the gear member 3102. The test tube is placed in the first mounting hole 3101. When the first driving component 32 drives the gear plate 34 to rotate to drive the gear member 3102 to rotate, the magnetic beads in the test tube are driven to rotate accordingly.

As shown in FIG. 3, the gear member 3102 includes a columnar main body 31024, which is provided with a first mounting hole 3101 running through two ends thereof. One end of the main body 31024 is provided with a gear tooth 31025 meshing with the gear plate 34. The gear tooth 31025 is provided on a peripheral side of the main body 31024. The wall of the first mounting hole 3101 is provided with a second mounting hole 31021. A magnetic member 33 is provided in the second mounting hole 31021, and the magnetic beads in the test tube are attracted by a magnetic attraction force of the magnetic member 33. When the gear plate 34 drives the main body 31024 to rotate, a position of the magnetic member 33 relative to the test tube in the first mounting hole 3101 moves with the rotation of the main body 31024, and the magnetic beads in the test tube are driven to rotate, thereby performing the nucleic acid extraction process. Preferably, the magnetic member 33 is a magnet.

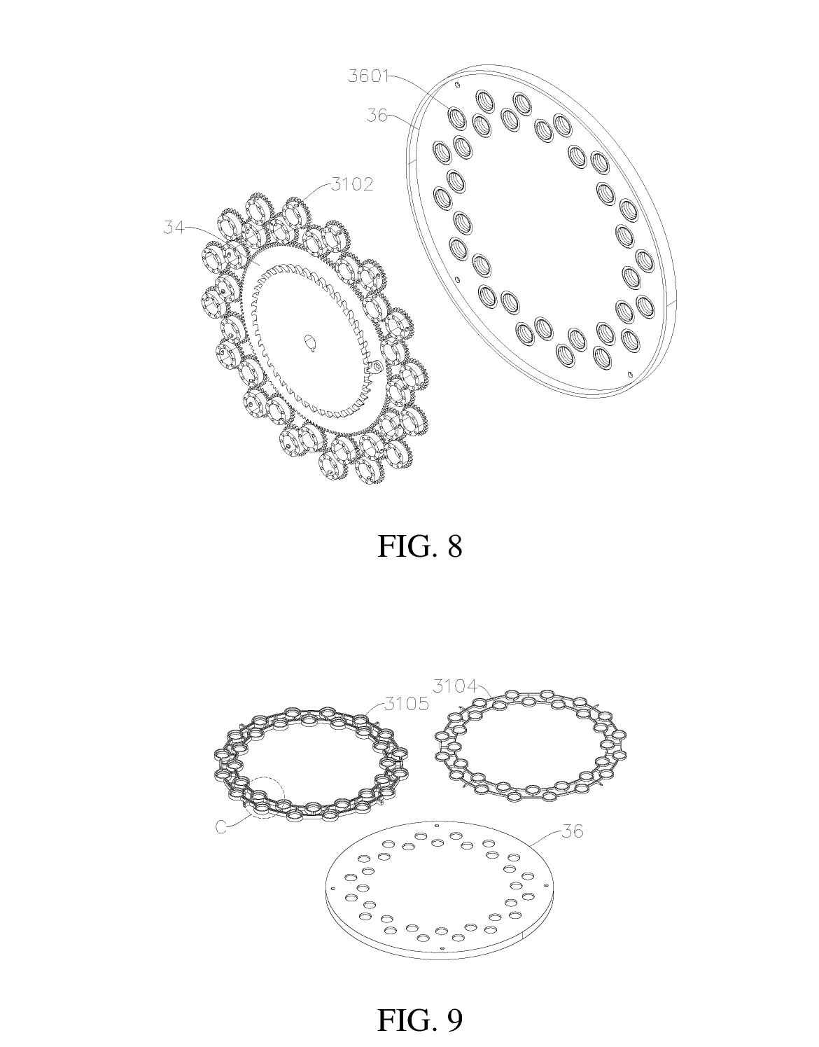

As can be seen from FIGS. 4-8, the extraction unit 3 further includes a bottom plate 35 and a top plate 36. An accommodating cavity is formed between the bottom plate 35 and the top plate 36. The gear plate 34 is arranged in the accommodating cavity. In this embodiment, a second through-hole 3502 is arranged on the bottom plate 35 for the output shaft of the first driving member 32 to pass through and connect to the gear plate 34. The bottom of the gear member 3102 is fitted to the bottom plate 35, and the top of the gear member 3102 is fitted to the top plate 36.

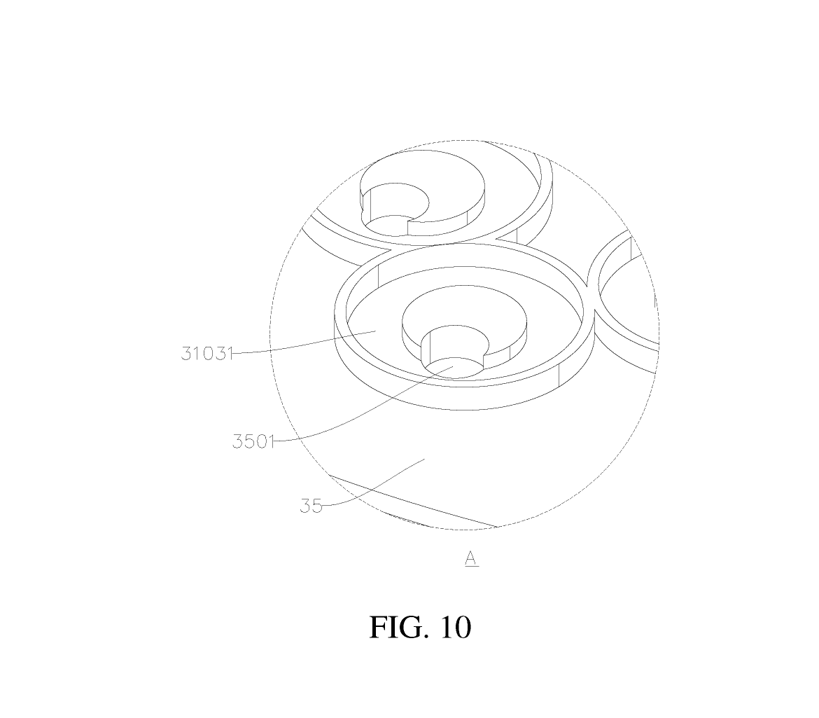

The bottom plate 35 in this embodiment is provided with a third mounting hole 3501 (as shown in FIG. 10) for the bottom of the test tube to pass through. In this way, the portion of the bottom of the test tube passing through the third mounting hole 3501 is exposed to the extraction unit 3, and this portion is located above the amplification unit 2. The amplification unit 2 performs temperature regulation, such as heating and cooling, on this portion to perform nucleic acid amplification.

It can be understood that bolt holes are provided at corresponding positions on the top plate 36 and the bottom plate 35, and the top plate 36 and the bottom plate 35 are connected by bolts. Alternatively, hollow mounting columns can be provided on the bottom plate 35, and bolts connect to the mounting columns by passing through the bolt holes of the top plate 36. The specific connection method is not limited here.

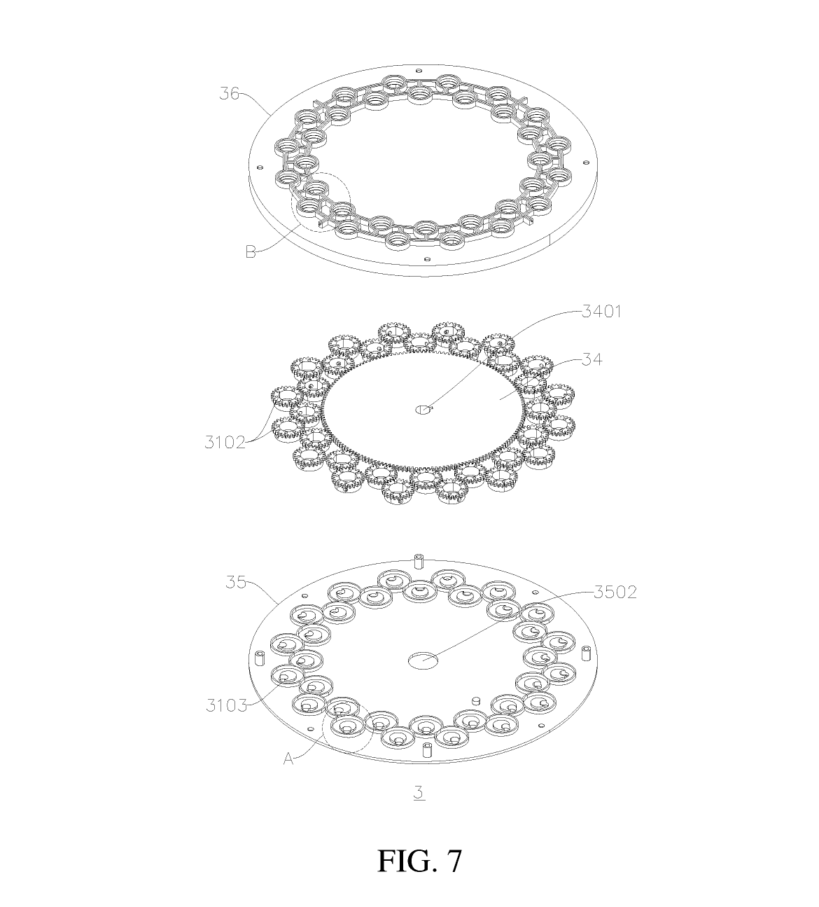

As can be seen from FIGS. 7, 8, and 10, the test tube mounting assembly 31 further includes a matching member 3103, which is arranged on the bottom plate 35. The gear member 3102 is arranged between the top plate 36 and the matching member 3103, and the gear member 3102 is arranged corresponding to the matching member 3103. In addition, an end surface of the gear member 3102 facing the bottom plate 35 is provided with a fourth mounting hole 31022, and a ball is arranged in the fourth mounting hole 31022. A side surface of the matching member 3103 facing the top plate 36 is provided with a first slide groove 31031, and the gear member 3102 can rotate in the first slide groove 31031 via the ball. When the gear member 3102 rotates, the ball revolves along a trajectory defined by a shape of the first slide groove 31031. In this embodiment, the matching member 3103 not only provides support for the mounting of the gear member 3102, but further prevents the gear member 3102 from moving unsmoothly when rotating. In this embodiment, the third mounting hole 3501 passes through the first slide groove 31031 is configured for the bottom of the test tube to run through.

It can be understood that the gear member 3102 is provided with a fifth mounting hole 31023 on an end surface facing the top plate 36 in this embodiment, and a ball is arranged in the fifth mounting hole 31023. A side surface of the top plate 36 facing the bottom plate 35 is provided with a second slide groove 3601 corresponding to the gear member 3102. The gear member 3102 can rotate in the second slide groove 3601 via the ball. When the gear member 3102 rotates, the ball rotates along a trajectory defined by a shape of the second slide groove 3601.

It should be noted that, in this embodiment, a plurality of fourth mounting holes 31022 are provided on a bottom of the gear member 3102 to balance the mounting height between various parts of the gear member 3102 and the mating piece 3103. In addition, a plurality of fifth mounting holes 31023 are provided on a top of the gear member 3102 to balance the mounting height between various parts of the gear member 3102 and the second slide groove 3601.

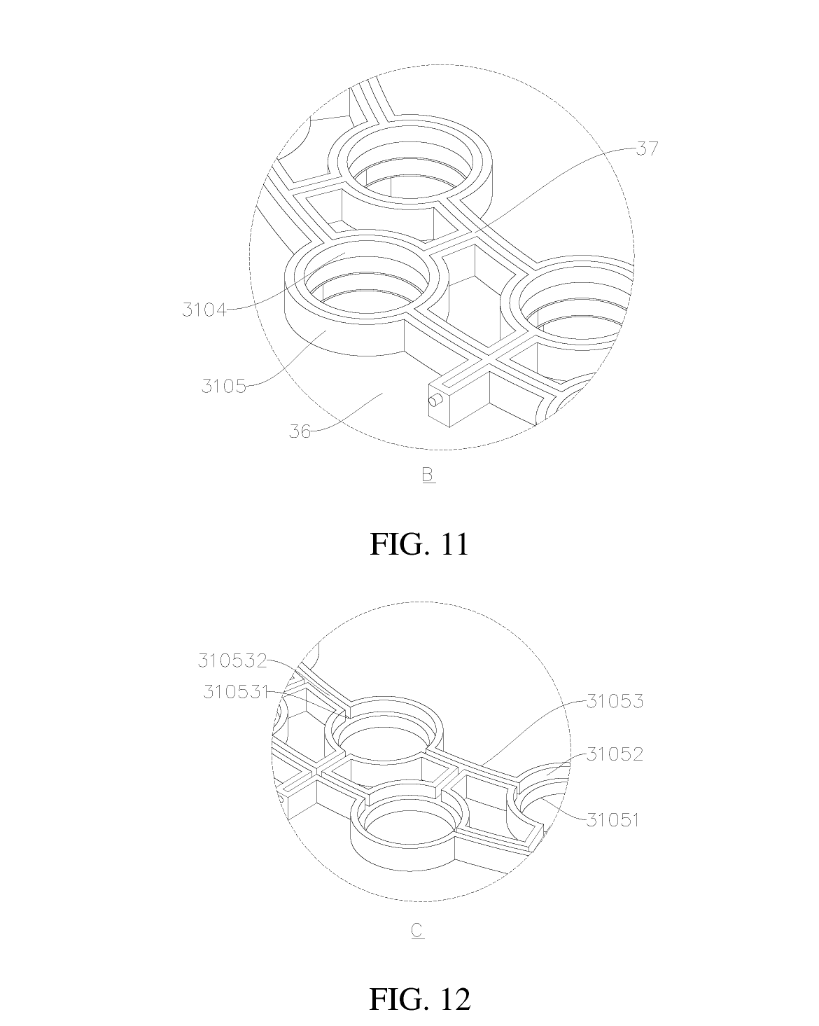

As can be seen from FIGS. 9 and 11, the test tube mounting assembly 31 further includes a first heating member 3104, which is arranged on the top plate 36 and corresponds to the gear member 3102. When the test tube is placed in the test tube mounting assembly 31, the test tube passes through the first heating member 3104, the first mounting hole 3101 and the third mounting hole 3501 in sequence, and a bottom of the test tube is exposed below the extraction unit 3, so that the light emitted from the fluorescence detection unit 4 directly reaches the bottom of the test tube. The first heating member 3104 is provided to heat a top of the test tube to improve the sensitivity of nucleic acid extraction. It can be understood that a through-hole is provided on the top plate 36, which is arranged corresponding to the second slide groove 3601 and the first heating member 3104 and is configured to allow the test tube to pass through.

As can be seen from FIGS. 9 and 11, the test tube mounting assembly 31 further includes a heat insulating member 3105. The heat insulating member 3105 is sleeved on an outer periphery of the first heating member 3104 to play a heat insulating role and prevent heat from diffusing toward the middle and bottom of the test tube.

As shown in FIG. 5, in some embodiments, a plurality of test tube mounting assemblies 31 are provided, and the plurality of the test tube mounting assemblies 31 are arranged around a peripheral side of the gear plate 34 to form a test tube mounting assembly group. This embodiment is described by taking the gear member 3102 as an example.

In this embodiment, the plurality of gear members 3102 are mounted on the peripheral side of the gear plate 34 in a circular arrangement, and the plurality of gear members 3102 are respectively meshed with the gear plate 34. In this way, mounting space can be saved, and the plurality of gear members 3102 can be driven to rotate synchronously by one gear plate 34, resulting in fewer transmission times and high efficiency. Furthermore, this arrangement prevents a situation where damage to one gear member 3102 affects the operation of the entire integrated device for nucleic acid extraction, amplification and detection, thereby enhancing the stability of the integrated device. On the other hand, the circular arrangement achieves a simpler and more compact structure, allowing the heating of the amplification device 2 to be more concentrated, achieving more uniform heating, resulting in a smaller temperature difference between adjacent test tubes, and improving uniformity. The number of the test tube mounting assembly groups is n, and the n test tube mounting assembly groups are sequentially arranged along a radial direction of the gear plate 34, where n ≥ 1.

When n is 1, the gear members 3102 are arranged around the peripheral side of the gear plate 34 to form a ring-like structure. When n is 2, the gear members 3102 are arranged around the peripheral side of the gear plate 34 to form a ring-like structure, which is an inner gear ring; a plurality of gear members 3102 are arranged around an outer side of the inner gear ring to form another ring-like structure, which is an outer gear ring. In this way, the outer gear ring is meshed with the inner gear ring, and the gear members of the inner gear ring can be driven to rotate by the rotation of the gear plate 34, and the gear members of the outer gear ring are driven to rotate accordingly, thereby achieving a synchronous rotation of the gear members of the inner gear ring and the outer gear ring.

It can be understood that the gear member 3102 of the outer gear ring is meshed with two adjacent gear members 3102 of the inner gear ring, that is, the gear members 3102 of the outer gear ring and the gear members 3102 of the inner gear ring are arranged in a staggered manner to achieve synchronous transmission. n can further be 3, 4 or more, and the arrangement of the gear members 3102 follows this pattern analogously. Preferably, n in this embodiment is 2, and each gear ring includes 16 gear members 3102, which can realize nucleic acid extraction and amplification on 32 samples at the same time, improve the efficiency of nucleic acid extraction and amplification, and realize the single module multi-throughput setting in structure.

Similarly, a plurality of matching members 3103 are arranged at intervals around an axis of the bottom plate 35, and the matching members 3103 are arranged in one-to-one correspondence with the gear members 3102. A plurality of second slide grooves 3601 are arranged at intervals around an axis of the top plate 36, and the second slide grooves 3601 are arranged in one-to-one correspondence with the gear members 3102. A plurality of first heating members 3104 are arranged at intervals around the axis of the top plate 36, and the first heating members 3104 are arranged in one-to-one correspondence with the gear members 3102. Here, the heat insulating members 3105 are arranged corresponding to the first heating members 3104, that is, a plurality of heat insulating members 3105 are arranged at intervals around the axis of the top plate 36, and the heat insulating members 3105 are sleeved on an outer periphery of the respective first heating members 3104 to play a heat insulating role and prevent the heat of the first heating member 3104 from diffusing to the middle and bottom of the test tube.

As shown in FIG. 12, in this embodiment, the heat insulating member 3105 includes a first base 31051 connected to the top plate 36 and a first mounting groove 31052 located on the first base 31051. The first base 31051 has a hollow structure, and an aperture of the first mounting groove 31052 is larger than an aperture of the hollow structure, so that when the first heating member 3104 is mounted on the heat insulating member 3105, it will not directly contact the top plate 36, preventing heat from diffusing to the middle and bottom of the test tube. Two adjacent heat insulating members 3105 are connected by a first connecting member 31053, and the first connecting member 31053 includes a second base 310531 and a second mounting groove 310532. The second base 310531 connects to the first base 31051, the second mounting groove 310532 is in communication with the first mounting groove 31052, and the second mounting groove 310532 is strip-shaped. It can be understood that in this embodiment, the heat insulating member 3105 and the first connecting member 31053 can be integrally formed or can be composed of an assembled structure. Similarly, two adjacent first heating members 3104 are connected by a second connecting member 37 (as shown in FIG. 11). The first heating member 3104 is mounted in the first mounting groove 31052, and the second connecting member 37 is mounted in the second mounting groove 310532.

It should be noted that the top plate 36 and the bottom plate 35 in this embodiment are preferably disc-shaped, to match disc-shaped structures formed by the test tube mounting assembly 31 and the gear plate 34.

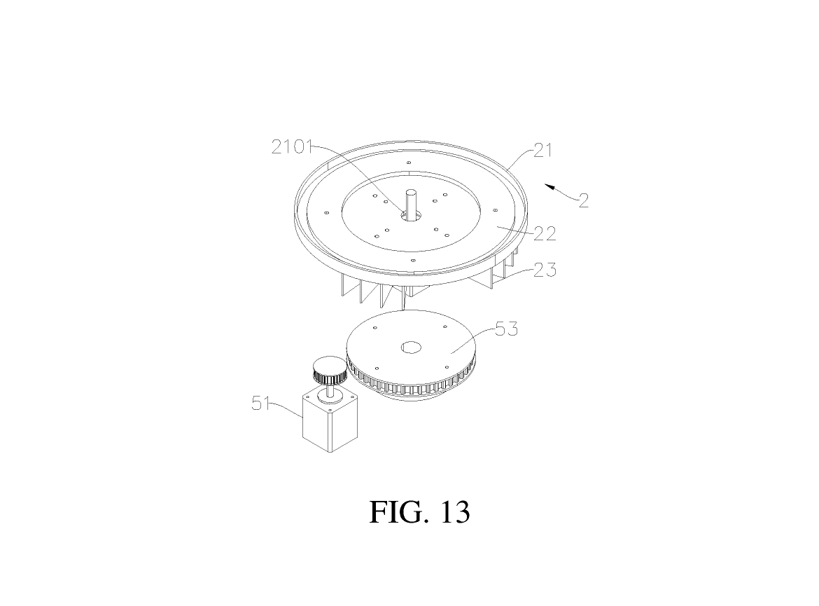

As can be seen from FIGS. 4, 13 and 14, the amplification unit 2 includes a heating plate 21, a second heating member 22 and a heat conductive member 23. The heating plate 21 is provided with a third through-hole 2101, so that an output shaft of the first driving member 32 can pass through the third through-hole 2101 of the heating plate 21 to connect with the extraction unit 3. The second heating member 22 is mounted on the heating plate 21 and is located below the extraction unit 3, so as to heat the bottom of the test tube. The heat conductive member 23 and the first driving member 32 are arranged on a side of the heating plate 21 away from the second heating member 22, and the heat conductive member 23 is configured to perform heat conduction and cooling to achieve PCR (Polymerase Chain Reaction) amplification.

In some embodiments, a plurality of the second heating members 22 are provided, and the plurality of the second heating members 22 are arranged around an axis of the heating plate 21 to form a ring-shaped structure.

It can be understood that the heating plate 21 has a disc-shaped structure, which is compatible with the structure of the bottom plate 35. In this embodiment, the plurality of the second heating members 22 are of the same size, and the annular structure formed by splicing the plurality of the second heating members 22 is compatible with the ring-shaped structure of the test tube mounting assembly 31, so as to heat the bottoms of the test tubes more concentratedly. In this embodiment, the heating plate 21 is formed with a side wall extending toward the bottom plate 35, which can prevent heat from dissipating to a certain extent.

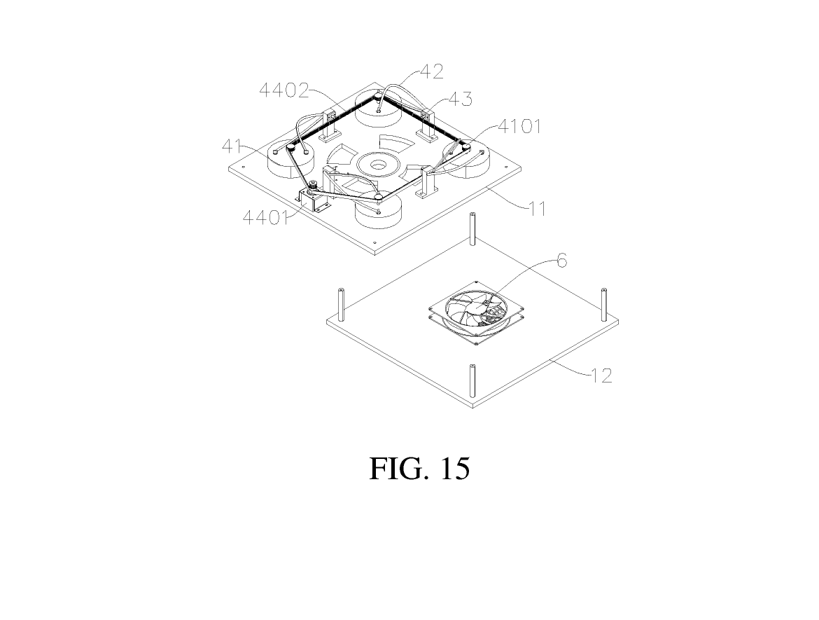

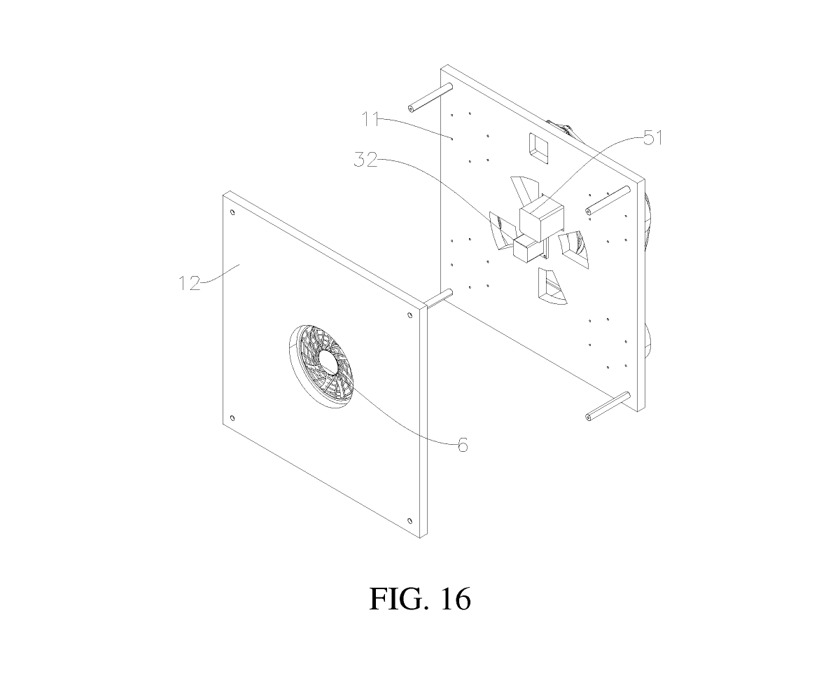

As can be seen from FIGS. 15 and 16, a cooling fan 6 is further included, which is mounted on the support 1. A suction port of the cooling fan 6 faces the amplification unit 2, and the cooling fan 6 is configured to suck heat and blow it out of the device, to improve the cooling capacity of the amplification unit 2. The cooling fan 6 cooperates with the second heating member 22 and the heat conductive member 23 to perform PCR amplification. It can be understood that a heat dissipation port is provided on the support 1. The heat dissipation port is located below the amplification unit 2, and is configured to allow the cooling fan 6 to suck the heat conducted by the heat conductive member 23 and dissipate the heat outside through the heat dissipation port.

In practical application, 1, 2, 3 or more cooling fans 6 may be provided according to actual needs.

It should be noted that the amplification process in this embodiment is a process of continuous heating and cooling cycles, including about 30-50 heating and cooling cycles, and fluorescence collection is required for each cycle. That is, there are 30 to 50 amplification cycles, and the fluorescence collection is performed 30 to 50 times.

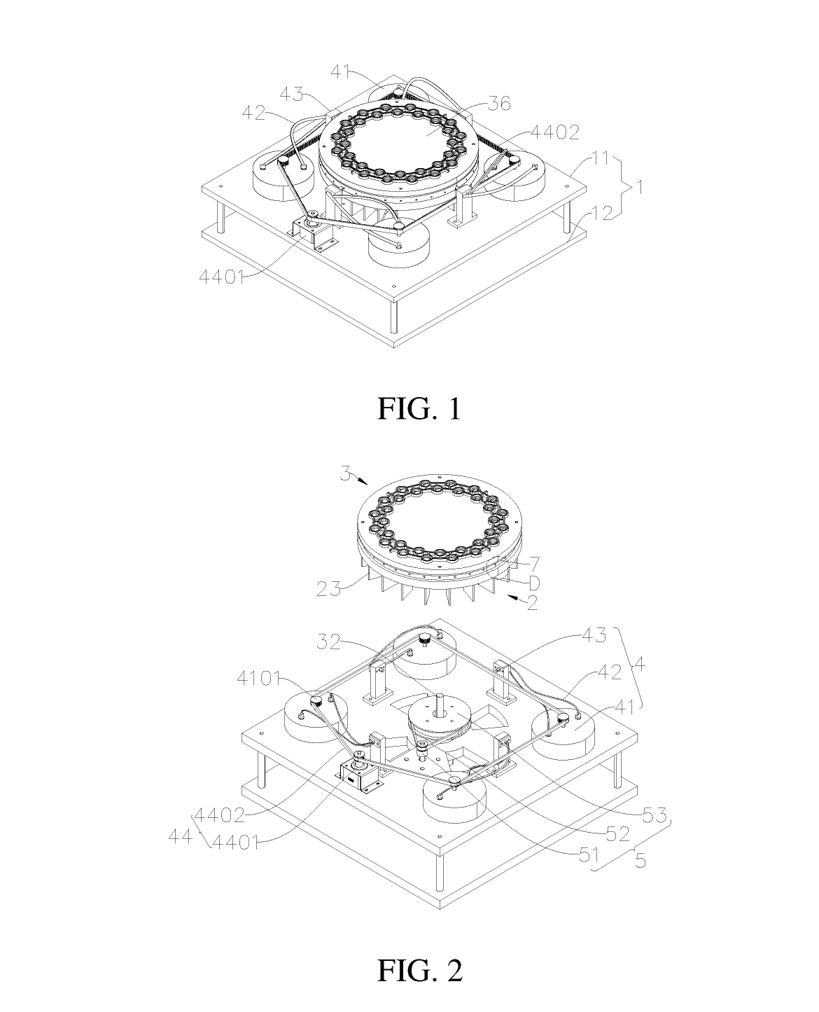

As can be seen from FIGS. 2 and 5, a light guiding unit 7 is disposed between the extraction unit 3 and the amplification unit 2. The light guiding unit 7 is provided with a sixth mounting hole 71 and a first light guiding hole 72. The sixth mounting hole 71 and the third mounting hole 3501 are arranged correspondingly, and the bottom of the test tube extends into the light guiding unit 7 through the sixth mounting hole 71. The light emitted from the fluorescence detection unit 4 is directed onto the bottom of the test tube through the first light guiding hole 72 of the light guiding unit 7, thereby performing fluorescence detection and collection for the nucleic acids. It can be understood that the light guiding unit 7 in this embodiment can not only play a light guiding role, but further play a certain light-shielding role after the test tube extends into the light guiding unit 7, to prevent the influence of external light.

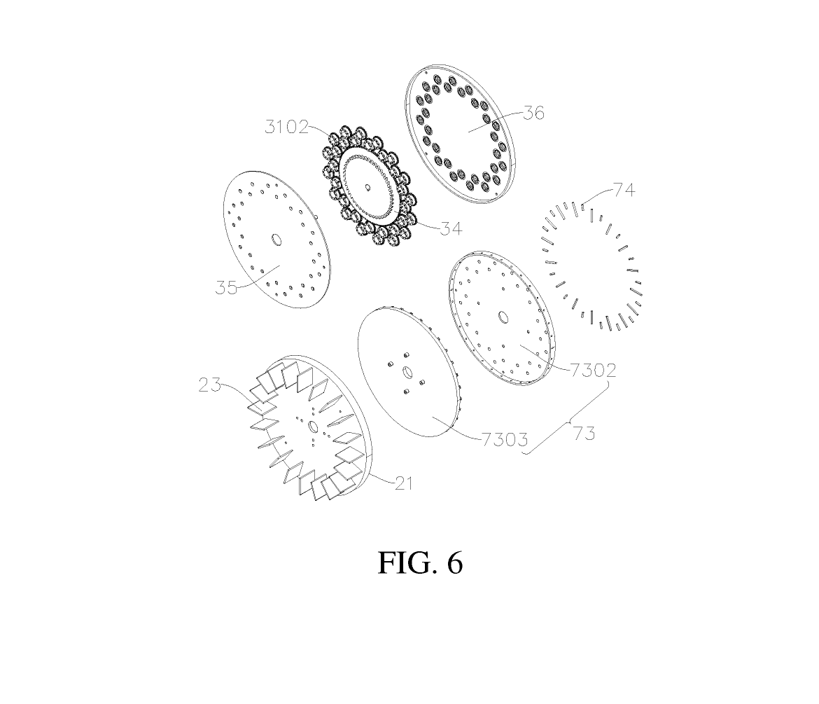

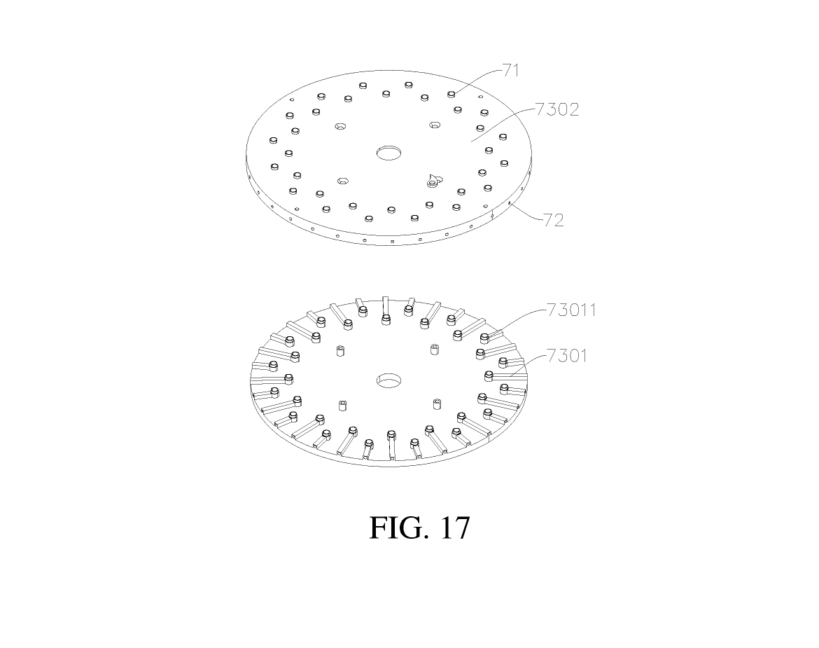

As can be seen from FIGS. 6 and 17, the light guiding unit 7 includes a light guiding plate 73 and a light guiding pillar 74. The first light guiding hole 72 is arranged on a side of the light guiding plate 73 facing the fluorescence detection unit 4, through which the light emitted from the fluorescence detection unit 4 passes. The sixth mounting hole 71 is arranged on a side of the light guiding plate 73 facing the extraction unit 3. The light guiding pillar 74 is arranged in the light guiding plate 73 and is located between the first light guiding hole 72 and the sixth mounting hole 71. The light guiding pillar 74 is configured to direct the light emitted from the fluorescence detection unit 4 onto the bottom of the test tube through the first light guiding hole 72.

Also, as can be seen from FIG. 6, in this embodiment, the light guiding plate 73 includes a first plate 7302 and a second plate 7303. The first plate 7302 and the second plate 7303 are connected to form a mounting cavity. The first plate 7302 is formed with a supporting wall extending toward the second plate 7303, the supporting wall is provided with the first light guiding hole 72, and the light guiding pillar 74 is mounted on the second plate 7303. During operation of the fluorescence detection unit 4, the light emitted from the fluorescence detection unit 4 enters through the first light guiding hole 72, and is transmitted via the light guiding pillar 74 to directly reach the bottom of the test tube, so as to realize fluorescence collection and feed the collected information back to the fluorescence detection unit 4, thereby performing nucleic acid fluorescence detection.

In some embodiments, the first light guiding hole 72 and the light guiding pillar 74 form an optical path for the light emitted from the fluorescence detection unit 4 to pass through. The center line of the first light guiding hole 72 is set at a certain angle to the center line of the sixth mounting hole 71, so that the light incident from the side can be directed onto the bottom of the test tube. Preferably, the center line of the first light guiding hole 72 is set perpendicular to the center line of the sixth mounting hole 71.

As shown in FIGS. 4 and 5, the light guiding plate 73 further includes a light guiding tube 7301, which is arranged on the second plate 7303. An end of the light guiding tube 7301 close to the first light guiding hole 72 is provided with a second light guiding hole. The second light guiding hole is in communication with the first light guiding hole 72. An end of the light guiding tube 7301 close to the sixth mounting hole 71 is provided with a light guiding part 73011 (as shown in FIG. 17), which is arranged in the sixth mounting hole 71. The light guiding part 73011 is provided with a through-hole to achieve communication with the sixth mounting hole 71. The light guiding pillar 74 is arranged in the light guiding tube 7301, so that the light emitted from the fluorescence detection unit 4 can be concentrated in the light guiding tube 7301, and the light directly illuminates the bottom of the test tube through the light guiding pillar 74, thereby improving the detection accuracy.

As shown in FIG. 17, in some embodiments, a plurality of the first light guiding holes 72 are provided, and the plurality of the first light guiding holes 72 are arranged on the supporting wall at intervals around the axis of the light guiding plate 73. A plurality of the sixth mounting holes 71 are provided, and the plurality of the sixth mounting holes 71 are arranged at intervals around the axis of the light guiding plate 73. It can be understood that in this embodiment, the plurality of the sixth mounting holes 71 are arranged on the first plate 7302 in a circular arrangement, corresponding to the position of the matching member 3103, so that the plurality of the sixth mounting holes 71 are in one-to-one correspondence with the plurality of the third mounting holes 3501, thereby performing nucleic acid fluorescence detection in the test tube. In this way, a distance between the sixth mounting hole 71 and a center of the first plate 7302 varies. The sixth mounting hole 71 corresponding to the third mounting hole 3501 located in the inner gear ring is defined as R1, and the distance from R1 to the center of the first plate 7302 is shorter. The sixth mounting hole 71 corresponding to the third mounting hole 3501 located in the outer gear ring is defined as R2, and the distance from R2 to the center of the first plate 7302 is longer. R1 and R2 are arranged alternatively, that is, one R2 is arranged between every two adjacent R1s, and they are arranged in one-to-one correspondence with the positions of the third mounting holes 3501. In some embodiments, if only one circle of matching members 3103 are provided, the distances between the sixth mounting holes 71 and the center of the first plate 7302 are all equal.

It should be noted that the first light guiding holes 72 and the sixth mounting holes 71 are arranged in a one-to-one correspondence, and one light guiding pillar 74 is disposed between each of the first light guiding holes 71 and a corresponding one of the sixth mounting holes 74.

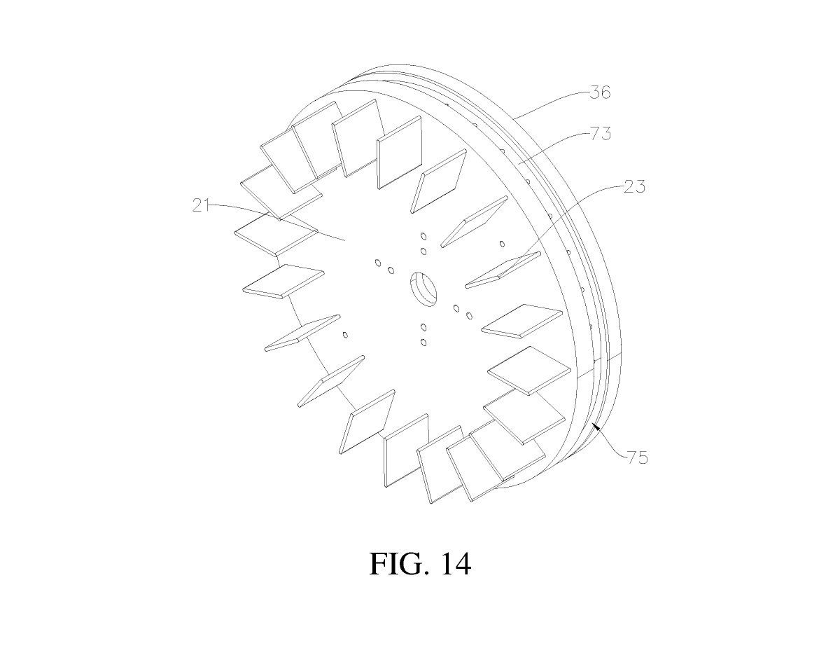

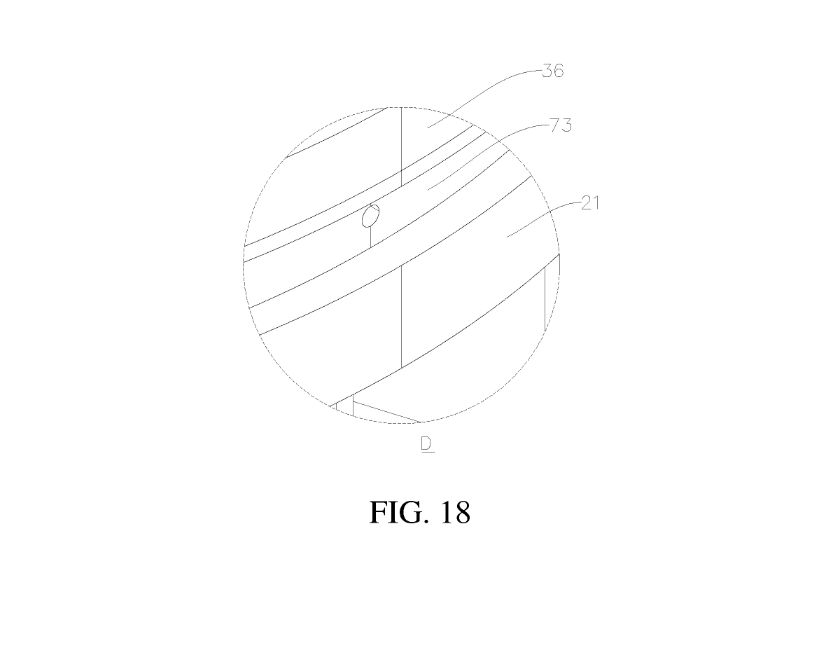

In some embodiments, the fluorescence detection unit 4 includes an emitting end. As shown in FIG. 18, a diameter of the light guiding plate 73 is smaller than a diameter of the extraction unit 3, and the diameter of the light guiding plate 73 is smaller than a diameter of the amplification unit 2, so that the extraction unit 3, the light guiding plate 73 and the amplification unit 2 define a groove 75 (as shown in FIG. 14). The emission end is located in the groove 75. During the light emission process of the emission end, the groove 75 can play a light-shielding role, so that the optical path will not be interfered with by external light, thereby preventing external light from significantly affecting the light emission and reception of the fluorescence detection unit 4, further avoiding impact on the detection results and making the detection results more accurate.

Also, as can be seen from FIG. 2, the fluorescence detection unit 4 includes a light emitting assembly 41, an optical fiber 42 and an optical fiber emitter 43. The light emitting assembly 41 is mounted on the support 1. The optical fiber emitter 43 is mounted on the support 1 through a mounting base. The optical fiber emitter 43 has an emission end, which can be also used as a reception end for receiving the detection results fed back from the test tube. The light emitting assembly 41 and the optical fiber emitter 43 are connected through two optical fibers 42. One optical fiber 42 is configured to connect the emission end of the light emitting assembly 41 and the emission end of the optical fiber emitter 43. The other optical fiber 42 is configured to connect the reception end of the light emitting assembly 41 and the reception end of the optical fiber emitter 43. The light emitted from the emission end of the optical fiber emitter 43 is directed onto the bottom of the test tube through the first light guiding hole 72 and the light guiding pillar 74.

As shown in FIG. 2, in some embodiments, a plurality of the light emitting assemblies 41 are provided, and the plurality of the light emitting assemblies 41 are arranged at intervals around an output shaft of the driving unit 5. A plurality of the optical fiber emitters 43 are provided, and the plurality of the optical fiber emitters 43 are arranged at intervals around an axis of the support 1.

In this embodiment, four light emitting assemblies 41 are provided, which are located at four corners of the support 1. Similarly, four optical fiber emitters 43 are provided. The optical fiber emitters 43 and the light emitting assemblies 41 are alternatively, that is, an optical fiber emitter 43 is arranged between two adjacent light emitting assemblies 41. By utilizing the gaps between the light emitting assemblies 41 to arrange the optical fiber emitter 43, the structure can be made more compact without occupying additional space.

As shown in FIG. 2, the fluorescence detection unit 4 further includes a driving assembly 44. The light emitting assembly 41 includes a plurality of light sources, a plurality of filters and a first transmission member 4101. The first transmission member 4101 is provided with a transmission end connected to the driving assembly 44 and a mounting end located on the emission optical path. The plurality of light sources are mounted on the mounting end at intervals around an axis of the mounting end, and each filter is correspondingly provided in front of an optical emission head of a corresponding light source.

It can be understood that in this embodiment, the light emitting assembly 41 is provided with different filters, and each light source and the corresponding filter cooperate in pairs to achieve the effect of outputting and receiving light with a preset wavelength band. Preferably, five kinds of filters are provided. The light source can emit light of five wavelength bands through the filters. In this embodiment, the mounting end of the first transmission member 4101 is fan-shaped. Five light sources are laid in sequence along the structural shape of the mounting end. The first transmission member 4101 is driven to rotate by the driving assembly 44. In response to that the driving assembly 44 drives the first transmission member 4101 to rotate once, the first transmission member 4101 drives a light source to rotate to the emission optical path to emit light with the corresponding wavelength. The light source emits monochromatic light through the filter, and the monochromatic light is transmitted to the optical fiber emitter 43 through the optical fiber 42, and illuminates the bottom of the test tube through the first light guiding hole 72 and the light guiding pillar 74 for performing fluorescence collection. The driving assembly 44 drives the first transmission member 4101 to switch different light sources and filters, and generates light of different colors in sequence until five fluorescence collections of the test tube are completed.

It can be understood that in this embodiment, the light source can be a laser or other types of light sources, which are not particularly limited herein, as long as they can be matched with corresponding filters in pairs to achieve the effect of outputting and receiving light with a preset wavelength band.

As shown in FIG. 2, in some embodiments, the driving assembly 44 includes a second driving member 4401 and a conveyor belt 4402, and the first transmission member 4101 and the second driving member 4401 are synchronously driven through the conveyor belt 4402. In this embodiment, the conveyor belt 4402 sequentially connects transmission ends of the plurality of first transmission members 4101, and the second driving member 4401 is configured to drive the plurality of the first transmission members 4101, so that the plurality of light sources are switched simultaneously to generate different monochromatic lights, thereby improving efficiency.

As shown in FIG. 2, the driving unit 5 includes a third driving member 51, a synchronous belt 52, and a second transmission member 53. The third driving member 51 is mounted on the support 1, and the second transmission member 53 is mounted on the bottom of the amplification unit 2. The third driving member 51 and the second transmission member 53 are synchronously driven by the synchronous belt 52 to drive the amplification unit 2 to rotate. It can be understood that the second transmission member 53 is sleeved over the output shaft of the first driving member 32, and the two do not contact each other.

After five fluorescence collections of the test tube are completed, the third driving member 51 drives the synchronous belt 52 to drive the second transmission member 53 to rotate, thereby driving the amplification unit 2 and the light guiding unit 7 located on the amplification unit 2 to rotate, so that the next first light guiding hole 72 is aligned with the emission end of the optical fiber emitter 43, then the above five-color fluorescence collection is performed again. The second transmission member 53 drives the light guiding unit 7 to rotate eight times in sequence, rotating a total of approximately 90°, to complete the fluorescence collection of the bottom of all test tubes in the extraction unit 3.

The device provided in this embodiment enables thirty-two samples to be placed into the device simultaneously and yield results together at the end, eliminating the need for manual detection, improving detection efficiency and shortening detection time. Structurally, it achieves single-module multi-throughput functionality. Standard quality control and blank control under the same conditions can be included in the thirty-two samples, which meets usage requirements and further enhances the efficiency and accuracy of the device. Additionally, detection throughput of the screening reagent can be improved.

Also, as can be seen from FIG. 15, the support 1 includes an top support 11 and a bottom support 12. The top support 11 and the bottom support 12 are connected by fasteners. The top support 11 and the bottom support 12 serve as a supporting platform to provide mounting space for the amplification unit 2, the extraction unit 3, the fluorescence detection unit 4, the driving unit 5 and the cooling fan 6. A space formed between the top support 11 and the bottom support 12 and the first driving member 32 and the third driving member 51 are located in the space. The first driving member 32, the third driving member 51, the amplification unit 2, and the fluorescence detection unit 4 are mounted on the top support 11, and the cooling fan 6 is mounted on the bottom support 12.

It can be understood that bolt holes are provided at corresponding positions on the top support 11 and the bottom support 12, and the two are connected by bolts. Alternatively, hollow mounting columns can also be provided on the bottom support 12, and bolts pass through the bolt holes of the top support 11 to connect to the mounting columns. The specific connection method is not limited here.

In summary, provides an integrated device for nucleic acid extraction, amplification and detection according to an embodiment of the present disclosure has a small size, facilitating transportation and portability. It not only reduces the difficulty of constructing and operating a laboratory but also solves the problem of aerosol contamination prone to occur in laboratories, thereby improving safety. Furthermore, it enables a single device to complete steps such as nucleic acid extraction, amplification, and detection, integrating multiple functions into one device.

It should be understood that terms such as "first" and "second" are used in the present disclosure to describe various information, but such information should not be limited by these terms. These terms are only used to distinguish information of the same type from each other. For example, without departing from the scope of the present disclosure, "first" information may also be referred to as "second" information, and similarly, "second" information may also be referred to as "first" information. Furthermore, terms such as "upper", "lower", "left", "right" and the like indicating orientations or positional relationships are based on the orientations or positional relationships shown in the drawings. They are merely for convenience of describing the present disclosure and simplifying the description, and do not indicate or imply that the referred device or unit must have a specific orientation, be constructed and operated in a specific orientation, and therefore should not be construed as limiting the present disclosure.

The foregoing descriptions are merely preferred embodiments of the present disclosure. It should be noted that for those of ordinary skill in the art, several improvements and substitutions may be made without departing from the technical principles of the present disclosure, and such improvements and substitutions should also be regarded as the protection scope of the present disclosure.

What is claimed is:

1. An integrated device for nucleic acid extraction, amplification and detection, comprising:
a support (1);
an amplification unit (2), disposed on the support (1), and configured to perform temperature regulation;
an extraction unit (3), mounted on the amplification unit (2), wherein the extraction unit (3) comprises a test tube mounting assembly (31), a first driving member (32) and a magnetic member (33), the test tube mounting assembly (31) is provided with a first mounting hole (3101), the magnetic member (33) is arranged in a wall of the first mounting hole (3101), and the first driving member (32) is mounted on the support (1), and configured to drive the test tube mounting assembly (31) to rotate, thereby driving the magnetic member (33) to rotate around an outer periphery of a test tube located in the test tube mounting assembly (31) and perform nucleic acid extraction;
a fluorescence detection unit (4), mounted on the support (1), and configured to perform fluorescence detection on the test tube; and
a driving unit (5), mounted on the support (1), wherein an output end of the driving unit (5) connects to the amplification unit (2), and is configured to drive the amplification unit (2) and the extraction unit (3) to rotate relative to the fluorescence detection unit (4).

2. The integrated device for nucleic acid extraction, amplification and detection according to claim 1, wherein the extraction unit (3) further comprises a gear plate (34), and an output shaft of the first driving member (32) passes through the amplification unit (2) and connects to the gear plate (34); and
the test tube mounting assembly (31) comprises a gear member (3102), wherein the gear member (3102) is provided with the first mounting hole (3101), the gear plate (34) is meshed with the gear member (3102), and the magnetic member (33) is arranged in the gear member (3102).

3. The integrated device for nucleic acid extraction, amplification and detection according to claim 2, wherein the first mounting hole (3101) passes through two ends of the gear member (3102), a second mounting hole (31021) is provided in the wall of the first mounting hole (3101), and the magnetic member (33) is arranged in the second mounting hole (31021).

4. The integrated device for nucleic acid extraction, amplification and detection according to claim 2, wherein the extraction unit (3) further comprises a bottom plate (35) and a top plate (36), an accommodating cavity is formed between the bottom plate (35) and the top plate (36), the gear plate (34) is arranged in the accommodating cavity, the bottom plate (35) is arranged on the amplification unit (2), and the bottom plate (35) is provided with a third mounting hole (3501) through which a bottom of the test tube passes.

5. The integrated device for nucleic acid extraction, amplification and detection according to claim 4, wherein an end surface of the gear member (3102) facing the bottom plate (35) is provided with a fourth mounting hole (31022), an end surface of the gear member (3102) facing the top plate (36) is provided with a fifth mounting hole (31023), and each of the fourth mounting hole (31022) and the fifth mounting hole (31023) comprises a ball;
the test tube mounting assembly (31) further comprises a matching member (3103), wherein the matching member (3103) is arranged on the bottom plate (35), a first slide groove (31031) is provided on a side of the matching member (3103) facing the top plate (36), and the third mounting hole (3501) passes through the first slide groove (31031); a second slide groove (3601) corresponding to the gear member (3102) is provided on a side of the top plate (36) facing the bottom plate (35);
the gear member (3102) is arranged between the top plate (36) and the matching member (3103), the gear member (3102) and the matching member (3103) are arranged correspondingly, and the gear member (3102) is configured to rotate in the first slide groove (31031) and the second slide groove (3601) via the balls.

6. The integrated device for nucleic acid extraction, amplification and detection according to claim 4, wherein the test tube mounting assembly (31) further comprises a first heating member (3104), and the first heating member (3104) is arranged on the top plate (36), corresponding to the gear member (3102); and
the test tube mounting assembly (31) further comprises a heat insulating member (3105), and the heat insulating member (3105) is sleeved on an outer periphery of the first heating member (3104).

7. The integrated device for nucleic acid extraction, amplification and detection according to claim 1, wherein a plurality of the test tube mounting assemblies (31) are provided, and the plurality of the test tube mounting assemblies (31) are arranged around an outer periphery of the gear plate (34) to form a test tube mounting assembly group;
wherein n test tube mounting assembly groups are provided and the n test tube mounting assembly groups are arranged in sequence along a radial direction of the gear plate (34), wherein n ≥ 1.

8. The integrated device for nucleic acid extraction, amplification and detection according to claim 1, wherein the amplification unit (2) comprises a heating plate (21), a second heating member (22) and a heat conductive member (23), the second heating member (22) is arranged on the heating plate (21) and located below the extraction unit (3), and the heat conductive member (23) and the first driving member (32) are arranged on a side of the heating plate (21) away from the second heating member (22); and
a plurality of the second heating members (22) are provided, and the plurality of the second heating members (22) are spliced around an axis of the heating plate (21) to form a ring.

9. The integrated device for nucleic acid extraction, amplification and detection according to claim 4, wherein a light guiding unit (7) is arranged between the extraction unit (3) and the amplification unit (2), the light guiding unit (7) is provided with a sixth mounting hole (71) and a first light guiding hole (72), the sixth mounting hole (71) and the third mounting hole (3501) are arranged correspondingly, the bottom of the test tube extends into the light guiding unit (7) through the sixth mounting hole (71), and light emitted from the fluorescence detection unit (4) passes through the first light guiding hole (72) and is directed onto the bottom of the test tube.

10. The integrated device for nucleic acid extraction, amplification and detection according to claim 9, wherein the light guiding unit (7) comprises a light guiding plate (73) and a light guiding pillar (74), the first light guiding hole (72) is arranged on a side of the light guiding plate (73) facing the fluorescence detection unit (4) to make the light emitted from the fluorescence detection unit (4) to pass through, the sixth mounting hole (71) is arranged on a side of the light guiding plate (73) facing the extraction unit (3), the light guiding pillar (74) is arranged in the light guiding plate (73) and is located between the first light guiding hole (72) and the sixth mounting hole (71), and the light guiding pillar (74) is configured to direct the light emitted from the fluorescence detection unit (4) onto the bottom of the test tube through the first light guiding hole (72).

11. The integrated device for nucleic acid extraction, amplification and detection according to claim 10, wherein a center line of the first light guiding hole (72) is arranged perpendicular to a center line of the sixth mounting hole (71); and
a plurality of the first light guiding holes (72) are provided, and the plurality of first light guiding holes (72) are arranged at intervals around an axis of the light guiding plate (73);
a plurality of the sixth mounting holes (71) are provided, the plurality of the sixth mounting holes (71) are arranged at intervals around the axis of the light guiding plate (73), and the plurality of the first light guiding holes (72) and the plurality of the sixth mounting holes (71) are arranged in a one-to-one correspondence;
the light guiding pillar (74) is disposed between each of the plurality of the first light guiding holes (72) and a corresponding one of the plurality of the sixth mounting holes (71).

12. The integrated device for nucleic acid extraction, amplification and detection according to claim 11, wherein the light guiding plate (73) further comprises a light guiding tube (7301), one end of the light guiding tube (7301) is in communication with the first light guiding hole (72), another end of the light guiding tube (7301) is in communication with the sixth mounting hole (71), and the light guiding pillar (74) is arranged in the light guiding tube (7301);
the fluorescence detection unit (4) comprises an emission end;
a diameter of the light guiding plate (73) is smaller than a diameter of the extraction unit (3), the diameter of the light guiding plate (73) is smaller than a diameter of the amplification unit (2), a groove (75) is defined among the extraction unit (3), the light guiding plate (73) and the amplification unit (2), and the emission end is located in the groove (75).

13. The integrated device for nucleic acid extraction, amplification and detection according to claim 12, wherein the fluorescence detection unit (4) comprises a light emitting assembly (41), an optical fiber (42) and an optical fiber emitter (43), the light emitting assembly (41) and the optical fiber emitter (43) are mounted on the support (1), the light emitting assembly (41) connects to the optical fiber emitter (43) through the optical fiber (42), the optical fiber emitter (43) is provided with the emission end, and light emitted from the emission end is directed onto the bottom of the test tube through the first light guiding hole (72) and the light guiding pillar (74);
a plurality of the light emitting assemblies (41) are provided, and the plurality of the light emitting assemblies (41) are arranged at intervals around an output shaft of the driving unit (5);
a plurality of the optical fiber emitters (43) are provided, and the plurality of the optical fiber emitters (43) are arranged at intervals around an axis of the support (1).

14. The integrated device for nucleic acid extraction, amplification and detection according to claim 13, wherein the fluorescence detection unit (4) further comprises a driving assembly (44), the light emitting assembly (41) comprises a plurality of light sources, a plurality of filters and a first transmission member (4101), the first transmission member (4101) is provided with a transmission end connected to the driving assembly (44) and a mounting end located on an emission optical path, the plurality of light sources are mounted on the mounting end at intervals around an axis of the mounting end, and each of the plurality of filters is correspondingly provided in front of an optical emission head of a corresponding one of the plurality of light sources;
in response to that the driving assembly (44) drives the first transmission member (4101) to rotate once, the first transmission member (4101) drives one of the plurality of light sources to rotate to the emission optical path to emit light with a corresponding wavelength.

15. The integrated device for nucleic acid extraction, amplification and detection according to claim 14, wherein the driving assembly (44) comprises a second driving member (4401) and a conveyor belt (4402), and the first transmission member (4101) and the second driving member (4401) are synchronously driven through the conveyor belt (4402).

16. The integrated device for nucleic acid extraction, amplification and detection according to claim 1, wherein the driving unit (5) comprises a third driving member (51), a synchronous belt (52) and a second transmission member (53), the third driving member (51) is mounted on the support (1), the second transmission member (53) is mounted on a bottom of the amplification unit (2), and the third driving member (51) and the second transmission member (53) are synchronously driven through the synchronous belt (52) to drive the amplification unit (2) to rotate.
